(19) United States
(12) Patent Application Publication
(10) Pub. No.: US 2010/0056869 A1
(43) Pub. Date: Mar. 4, 2010

(54) ENDOSCOPE

(75) Inventors: Kazuhito HAYAKAWA, Tokyo (JP); Yoshimasa KUSAZAKI, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: OLYMPUS CORPORATION, Tokyo (JP)

(21) Appl. No.: 12/546,972

(22) Filed: Aug. 25, 2009

(30) Foreign Application Priority Data
Aug. 27, 2008 (JP) 2008-218295

(51) Int. Cl. A61B 1/06

(52) U.S. Cl. 600/180

(57) ABSTRACT

An endoscope, at least a part of which is introduced into an object to be observed to image an internal subjects includes; an illuminating device which illuminates the front side and is disposed so that a normal line M is perpendicular to an introduction direction (X direction); and an imaging unit which images the front side and is disposed parallel to the illuminating device. The illuminating device includes an organic light emitting layer which is interposed between two electrodes, a light shielding portion which blocks first light proceeding in a direction of the normal line among the light generated from the organic light emitting layer, and a light extracting portion which is provided at the tip end of the organic light emitting layer and extracts second light proceeding in an in-plane direction of the organic light emitting layer from the tip end among the generated light to emit the second light to the front side.

### BACKGROUND OF THE INVENTION

1. Field of the Invention

The present invention relates to an endoscope which is introduced into an object to be observed to image the inside. Priority is claimed on Japanese Patent Application No. 2008-2182955 filed on Aug. 21, 2008, the content of which is incorporated herein by reference.

2. Description of the Related Art

For endoscopes of this type, it is important to brightly illuminate a subject in order to clearly image the subject. As one type of endoscope, there are known medical endoscopes for imaging the inside of a body cavity, and the endoscopes brightly illuminate a subject in a body cavity by introducing light with the use of an optical cable from a light source device disposed outside the body cavity. This configuration is generally well known.

In recent years, capsule-type endoscopes (capsule endoscopes) capable of being swallowed have been developed.

Since the capsule endoscopes can image the inside of a body cavity while markedly reducing the burden, on a human subject, the capsule endoscopes are an example of an endoscope attracting attention. In the case of capsule endoscopes, an illuminating device provided in a capsule-like casing illuminates a subject.

In general, an LED disposed near an imaging unit serves as the illuminating device. Also, there are known capsule endoscopes in which in place of an LED, an organic EL element as an organic light emitting diode (OLED) including a planar light emitting layer made of an organic compound is used as an illuminating device (for example, see JP-A-2006-255247). The capsule endoscopes are configured so that the organic EL element is disposed near an imaging unit to perform an illuminating operation by surface emission in a direction of view of the imaging unit.

In particular, the organic EL element is a thin film device having a very thin thickness, in which a transparent electrode functioning as an anode, an organic light emitting layer and a metal thin film functioning as a cathode are laminated on a transparent substrate in order. Accordingly, the organic EL element is preferably used as an illuminating device because the organic EL element can more contribute to the miniaturization of capsule endoscopes than an LED.

Incidentally, as long as an organic EL element is used as an imaging device, it is necessary to illuminate a subject as brightly as possible. In particular, in the case of capsule endoscopes, the illumination which is performed by outside light as in cameras cannot be obtained. Accordingly, only the light emitted from an illuminating device provided in a casing becomes illuminating light, so the organic EL element is required to illuminate a subject as brightly as possible.

The organic EL element described in JP-A-2006-255247 is formed to be perpendicular to a center axis of the capsule endoscope and to spread radially in order to use the light emitted from the surface as illuminating light. The organic EL element emits light in a normal surface direction (lamination direction) to perform an illuminating operation in a direction of view of the imaging unit.

In particular, the light emitted from the organic EL element in the normal direction is about 30% of the light generated by the organic light emitting layer. The remaining 70% of the light propagates in an in-plane direction and is not emitted in the normal direction.

Accordingly, the organic EL element is formed to markedly spread radially in order to ensure a light emission area as large as possible, in this manner, the organic EL element sufficiently illuminates a subject and ensures the intensity of light necessary for imaging.

### SUMMARY OF THE INVENTION

An object of the invention is to provide an endoscope which can ensure the sufficient intensity of light necessary for imaging and in which a reduction in diameter can be achieved.

In order to achieve the object, the invention provides the following means.

An endoscope according to the invention, at least a part of which is introduced into an object to be observed to image an internal subject, is disposed so that a normal line is perpendicular to an introduction direction. The endoscope according to the invention includes an illuminating device which illuminates the front side and an imaging unit which images the front side and is disposed parallel to the illuminating device. Further, the illuminating device includes an organic light emitting layer which is interposed between two electrodes, a light shielding portion which blocks first light proceeding in a direction of the normal line among the light generated from the organic light emitting layer, and a light extracting portion which is provided at the tip end of the organic light emitting layer and extracts second light proceeding in an in-plane direction of the organic light emitting layer from the tip end among the generated light to emit the second light to the front side.

In the endoscope according to this embodiment, when a voltage is applied to the two electrodes, the organic-light emitting layer generates light. At this time, about 30% of the generated light becomes the first light proceeding in the direction of the normal line (direction perpendicular to introduction direction) perpendicular to the surface of the organic light emitting layer. The remaining amount, roughly 70%, is the second light proceeding in the in-plane direction (introduction direction) of the organic light emitting layer. Among them, the first light proceeding in the direction of the normal line is blocked by the light shielding portion or reflected to become the second light proceeding in the in-plane direction. Meanwhile, the second light proceeding in the in-plane direction is extracted by the light extracting portion provided at the tip end of the organic light emitting layer to be emitted to the front side. In this manner, in introducing the endoscope into the object to be observed, the front side is illuminated. That is, an illuminating operation is performed in a direction of view of the imaging unit, and a subject is thus brightly illuminated. As a result, the subject can be imaged by the imaging unit.

In particular, the light intensity of the second light proceeding in the in-plane direction of the organic light emitting layer is larger than that of the first light proceeding in the direction of the normal line. So, unlike in the case of conventional surface emission devices emitting light from the surface with the use of the first light as the illuminating light, sufficient intensity of light necessary for imaging can be ensured.

Unlike conventional devices which emit light from the surface, the illuminating device is disposed so that the normal line is perpendicular to the introduction direction. Accordingly, the diameter of the endoscope can be reduced.

In the endoscope according to the invention, the imaging unit may be disposed to protrude further forward from the illuminating device than the light extracting portion.

Since the imaging unit protrudes further forward from the illuminating device than the light extracting portion in the endoscope according to the invention, the front side can be illuminated by emitting the second light from behind the imaging unit. Accordingly, it is difficult for the second light to enter the imaging unit. So, the deterioration of a taken image caused due to unnecessary light entering the imaging unit can be prevented. Therefore, a subject can be more clearly imaged and imaging performance can be improved.

In the endoscope according to the invention, the imaging unit may be disposed to retreat further backward from the illuminating device than the light extracting portion.

Since the imaging unit retreats further backward from the illuminating device than the light extracting portion in the endoscope according to the invention, the second light can be emitted so as to illuminate the front side of the imaging unit. Accordingly, the imaging unit does not block the optical path of the second light as illuminating light, thereby not making a shadow. So, the endoscope according to the invention can securely brightly illuminate the front side and thus can more clearly image a subject. Therefore, imaging performance is improved.

In the endoscope according to the invention, the illuminating device may include a side light extracting portion for extracting the first light to emit the first light to the side.

In the endoscope according to the invention, since the side light extracting portion extracts the blocked or reflected first light and emits the first light to the side, the periphery of the visual field of the imaging unit can be directly or indirectly illuminated. Therefore, the endoscope according to the invention can increase the brightness of the whole visual field of the imaging unit and thus can more clearly image a subject. Since the side light extracting portion emits the first light to the side, the light intensity of the illuminating light (second light) illuminating the front side is not affected.

The endoscope according to the invention may further include a side imaging unit for imaging an area illuminated with the first light.

The endoscope according to the invention can image an area illuminated with the first light emitted from the side by the side imaging unit. Accordingly, the endoscope according to the invention can simultaneously image different visual fields by-cooperating with the imaging unit imaging the front side. Accordingly, in the endoscope according to the invention, the number of components can be reduced without the need for a separate arrangement of the front and side illuminating elements.

In the endoscope according to the invention, the organic light emitting layer may be formed into a cylindrical shape so as to surround the imaging unit.

Since the organic light emitting layer is formed into a cylindrical shape in the endoscope according to the invention, it is possible to increase the light intensity of the illuminating light (second light) illuminating the front side while maintaining a reduction in the diameter.

Accordingly, the endoscope according to the can more brightly illuminate a subject and thus can more clearly image the subject.

In the endoscope according to the invention., transparent covers for sealing the inside of the organic light emitting layer may be provided at opposite ends of the organic-light emitting layer to form the endoscope into a capsule shape as a whole.

Since the whole endoscope according to the invention is formed into a capsule shape, it can be used as a medical capsule endoscope.

In the endoscope according to the invention, the light extracting portion may also be provided at the base end of the organic light emitting layer to extract the second light from the base end and thereby emit the second light to the rear side, and a rear imaging unit for imaging an area illuminated at the rear side may be provided in the organic light emitting layer.

Since the light extracting portion emits the second light to the rear side as well as the front side, the endoscope according to the invention can illuminate both of the front side and the rear side at the same time. In addition, the imaging unit images the brightly illuminated front side and the rear imaging unit images the brightly illuminated rear side. Accordingly, in the endoscope according to the invention, the number of components can be reduced without the need for a separate arrangement of the front and rear illuminating elements.

### BRIEF DESCRIPTION OF THE DRAWINGS

FIG. 1 is an overall view of an endoscope according to a first embodiment of the invention.

FIG. 2 is a cross-sectional diagram of the endoscope shown in FIG. 1, taken along the line A-A.

FIG. 3 is a cross-sectional diagram in which an organic EL element of an illuminating device in the endoscope shown in FIG. 1 is enlarged.

FIG. 4 is a diagram showing a modified example of the endoscope according to the first embodiment and is a cross-sectional diagram of the endoscope in which an imaging unit is disposed to protrude further forward than the illuminating device.

FIG. 5 is a diagram showing a modified example of the endoscope according to the first embodiment and is a cross-sectional diagram of the endoscope in which the imaging unit is disposed to retreat further backward than the illuminating device.

FIG. 6 is a cross-sectional diagram of an endoscope according to a second embodiment of the invention.

FIG. 7 is a cross-sectional diagram showing a modified example of the endoscope according to the second embodiment.

FIG. 8 is a cross-sectional diagram of an endoscope according to a third embodiment of the invention.

### DETAILED DESCRIPTION OF THE INVENTION

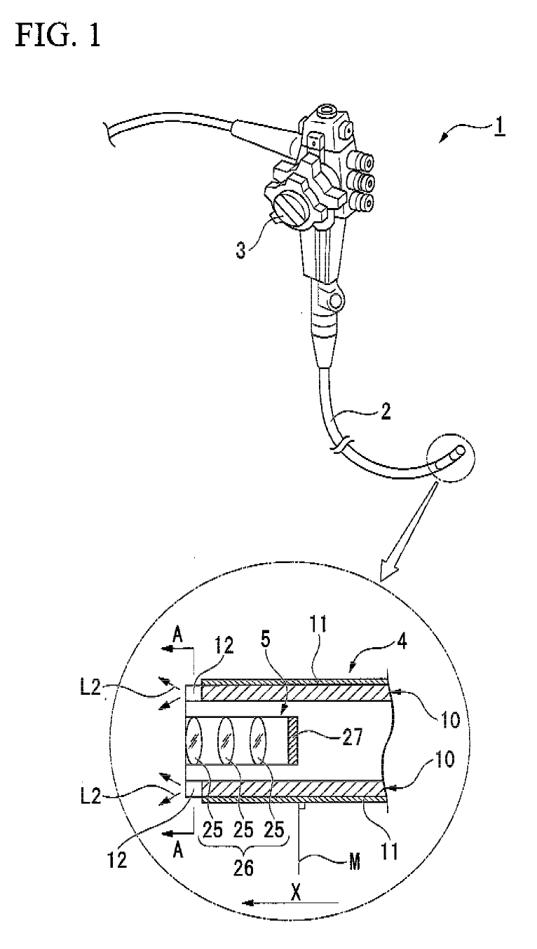

Hereinafter, a first embodiment of the invention will be described with reference to FIGS. 1 to 3. In this embodiment, a medical endoscope will be exemplified and described as an example of an endoscope.

As shown in FIG. 1, an endoscope 1 according to this embodiment is a member, at least a part of which is introduced into the body cavity (in the object to be observed) (not shown) of a human subject to image an internal subject (digestive tract, etc). The endoscope 1 mainly includes an insertion portion 2 which is inserted into a body cavity and an operation portion 3 which operates the insertion portion 2 so as to bend the insertion portion 2.

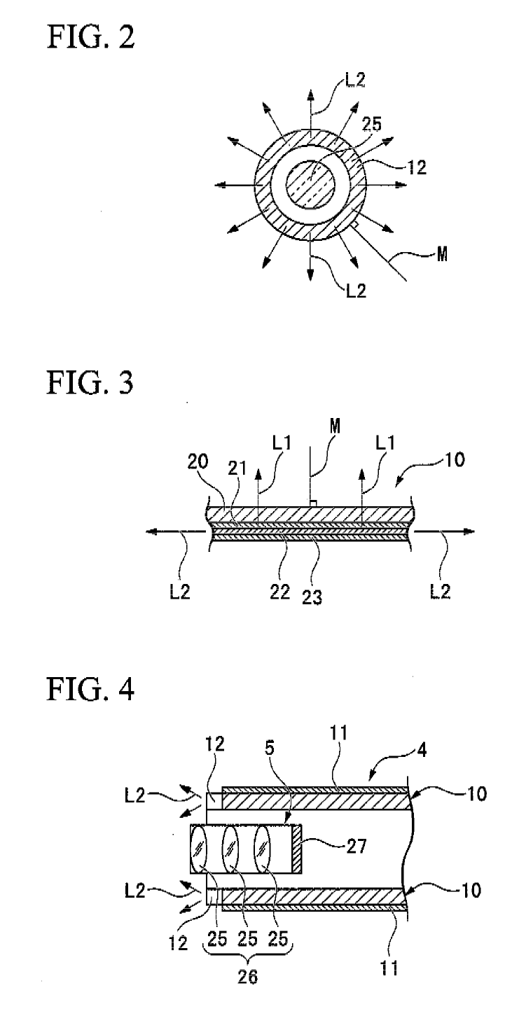

An illuminating device 4 for illuminating the front side and an imaging unit 5 for imaging the front side are provided at the tip end of the insertion portion 2 while being disposed parallel to each other. As shown in FIGS. 1 and 2, the illuminating device 4 is composed of an organic EL element 10 including an organic light emitting layer 22, a light shielding film (light shielding portion) 11 and a light extracting portion 12 and is formed into a cylindrical shape so as to surround the imaging unit 5. In addition, the illuminating device 4 is disposed so that a normal line M, perpendicular to the peripheral surface, is perpendicular to an introduction direction (the direction of the arrow X).

As shown in FIG. 3, the organic EL element 10 includes an anode 21 laminated on a transparent substrate 20, the organic light emitting layer 22 laminated on the anode 21 and a cathode 23 laminated on the organic light emitting layer 22. That is, the organic light emitting layer 22 is interposed between the two electrodes 21 and 23.

The transparent substrate 20 is, for example, an optically transparent substrate formed of a glass plate, a resin plate, a film, or the like. The anode 21 is a transparent electrode and is formed of, for example, a transparent conductive film of ITO, ATO, ZnO, or the like. The cathode 23 is formed of, for example, a transparent conductive film of ITO, ATO, ZnO, or the like, or a metal thin film such as an Al film, or the like. The organic light emitting layer 22 is obtained by laminating various materials necessary for light emission, and emits light by the combination of holes and electrons when a voltage is applied between the anode 21 and the cathode 23.

About 30% of the light generated at this time becomes the first light L1 passing through the anode 21 and the transparent substrate 20 and proceeding in a direction of the normal line M, and about 70% thereof becomes the second light L2 proceeding in an in-plane direction of the organic light emitting layer 22, due to the difference in refractive index. The organic EL element 10 according to this embodiment is formed into a cylindrical shape so that the transparent substrate 20 is positioned at the peripheral side. Accordingly, the first light proceeds to the side of the illuminating device 4.

As shown in FIG. 1, the light shielding film 11 is formed on the peripheral surface of the organic EL element 10 and blocks the first light L1 proceeding in the direction of the normal line M. Moreover, as shown in FIGS. 1 and 2, the light extracting portion 12 is provided at the tip end of the organic EL element 10 to extract the second light L2, proceeding in the in-plane direction of the organic light emitting layer 22, from the tip end and thereby emit the second light to the front side. In this manner, the illuminating device 4 can illuminate the front side of the introduced insertion portion 2 as described above.

As shown in FIG. 1, the imaging unit 5 is composed of an objective lens group 26 consisting of plural lenses 25 and a solid-state imaging element 27 disposed behind the objective lens group 26. The imaging unit 5 is disposed so that the tip end of the illuminating device 4 is flush with the tip end of the imaging unit 5.

The case will be described in which imaging is performed by using the endoscope 1 having the above-described configuration.

First, the insertion portion 2 is introduced into a body cavity of a human subject and proceeds while being operated and bent by the operation portion 3.

At this time, a voltage is applied between the anode 21 and the cathode 23 of the illuminating device 4. In this case, the organic light emitting layer 22 emits light. About 30% of this light becomes the first light L1 proceeding in the direction of the normal line M, and the remaining amount, roughly 70%, becomes the second light L2 proceeding in the in-plane direction.

Among them, the first light L1 proceeding in the direction of the normal line M is blocked by the light shielding film 11. The second light L2 proceeding in the in-plane direction is extracted by the light extracting portion 12 and emitted to the front side as shown in FIGS. 1 and 2. In this manner, in the endoscope 1, in introducing the insertion portion 2 into the body cavity, it is possible to perform an illuminating operation in a direction of view of the imaging unit 5. Accordingly, the endoscope 1 can brightly illuminate a digestive tract as a subject. As a result, the endoscope 1 can image the digestive tract as a subject by the imaging unit 5. Therefore, observation using the endoscope 1 is securely performed in the body cavity.

In particular, the light intensity of the second light L2 proceeding in the in-plane direction of the organic light emitting layer 22 is larger than that of the first light L1 proceeding in the direction of the normal line M. So, unlike conventional surface emission devices using the first light L1 as illuminating light, this device can ensure the sufficient intensity of light necessary for imaging. In this embodiment, since the illuminating device 4 is cylindrical, a subject can be more brightly illuminated and clearly imaged.

In addition, unlike the conventional devices emitting light from the surface, the illuminating device 4 is disposed so that the normal line M is perpendicular to the introduction direction. Accordingly, a dead space in the direction perpendicular to the introduction direction can be reduced and the diameter of the insertion portion 2 of the endoscope 1 can be reduced.

As described above, the endoscope 1 according to this embodiment can ensure that a light intensity sufficient to perform imaging is provided and thus a subject can be clearly imaged. Moreover, since the diameter of the endoscope 1 can be reduced, the burden on the human subject can also be reduced.

In the above embodiment, an example has been described in which the endoscope 1 includes at least the illuminating device 4 and the imaging unit 5. However, another configuration may be incorporated to function as the endoscope 1. For example, a treatment instrument channel for inserting a treatment instrument, an air line, a water line or the like, may be incorporated into the endoscope 1.

In the above embodiment, the imaging unit 5 and the illuminating device 4 are disposed so as to be flush with each other. However, as shown in FIG. 4, the imaging unit 5 may be disposed to protrude further forward from the illuminating device 4 than the light extracting portion 12.

Due to such a configuration, the second light L2 as illuminating light is emitted from behind the imaging unit 5 so as to illuminate the front side. Accordingly, in the above-described configuration, it is difficult for the second light L2 to turn around and enter the imaging unit 5. So, the above-described configuration can prevent the deterioration of a taken image caused due to unnecessary light entering the imaging unit. Therefore, the above configuration can more clearly image a subject and can improve imaging performance.

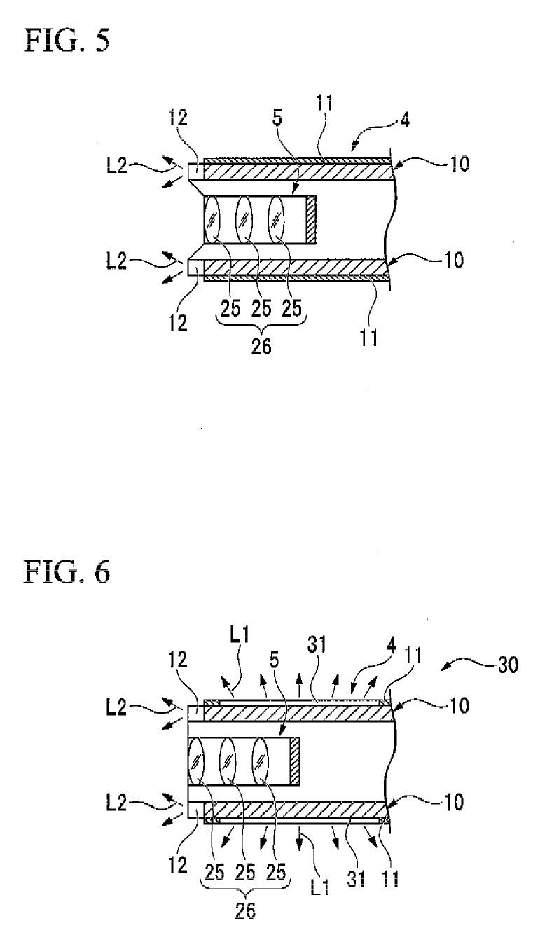

Moreover, on the contrary, as shown in FIG. 5, the imaging unit 5 may be disposed to retreat further backward from the illuminating device 4 than the light extracting portion 12.

Due to such a configuration, the second light L2 as illuminating light is emitted so as to illuminate the front side of the imaging unit 5. Accordingly, in the above-described configuration, the imaging unit 5 does not block the second light L2, thereby not making a shadow. So, the above-described configuration can securely i brightly illuminate the front side and thus can more clearly image a subject. Therefore, the above configuration can have improved imaging performance.

Next, a second embodiment according to the invention will be described. In embodiment, the same portions as the constitute elements in the first embodiment are denoted by the same reference numerals and a description thereof will be omitted.

A difference between the first embodiment and the second embodiment is as follows. In the first embodiment, the illuminating device 4 illuminates only the front, but in the second embodiment, the illuminating device 4 performs a simultaneous illuminating operation of the front and the side.

i That is, in an endoscope 30 according to this embodiment, an illuminating

device 4 includes a side light extracting portion 31 for extracting a first light L1 proceeding in a direction of a normal line M and emitting the first light to the side, as shown in FIG. 6. Accordingly, in this embodiment, the side light extracting portion 31 extracts the blocked first light L1 to emit the first light to the side. So, the endoscope 30 according to this embodiment can directly or indirectly illuminate the periphery of the visual field of an imaging unit 5. Therefore, the brightness of the whole visual field of the imaging unit 5 is increased more than in the first embodiment and a subject can be more clearly imaged.

In addition, in the endoscope 30 according to this embodiment, the first light L1 is emitted only to the side, so the light intensity of illuminating light (second light L2) irradiating the front side is not affected.

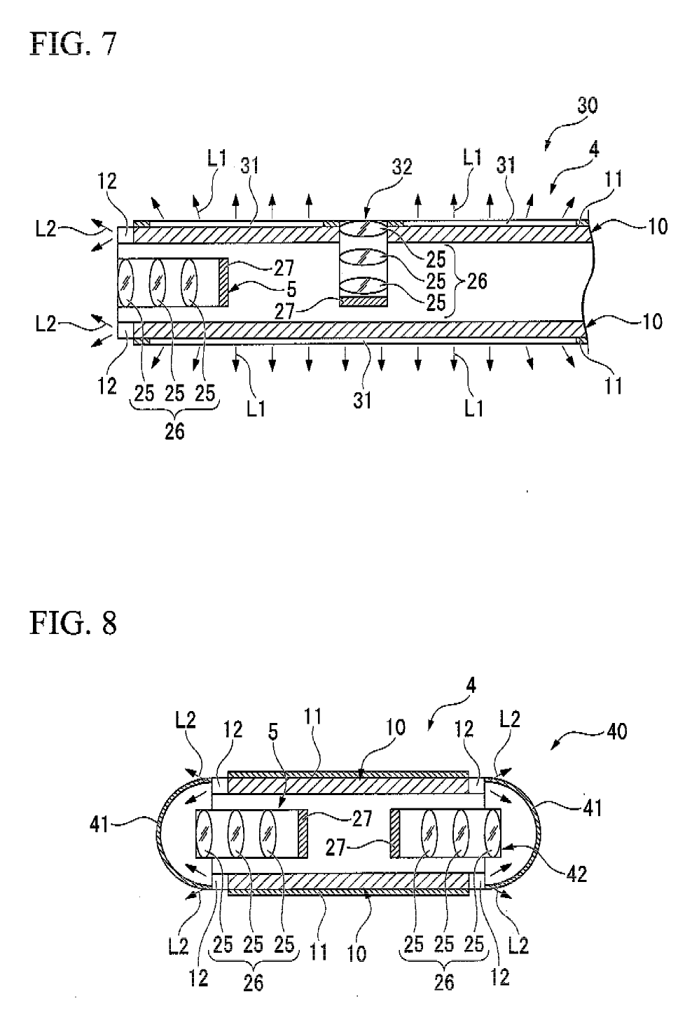

In this embodiment, as shown in FIG. 7, the endoscope 30 may include a side imaging unit 32 for imaging an area which is illuminated with the first light L1 emitted toward the side. The side imaging unit 32 is composed of an objective lens group 26 and a solid-state imaging element 27 as in the imaging unit 5.

In this case, different visual fields can be simultaneously imaged by allowing the cooperation of the side imaging unit 32 and the imaging unit 5. Accordingly, in the endoscope 30 according to this embodiment, the number of components can be reduced without the need for a separate arrangement of the front and side illuminating elements.

Next, a third embodiment according to the invention will be described. In this third embodiment, the same portions as the constitute elements in the first embodiment are denoted by the same reference numerals and a description thereof will be omitted.

A different thing between the first embodiment and the third embodiment is as follows. The endoscope according to the first embodiment is a general device in which only the insertion portion 2 is introduced into the body cavity of a human subject, but the endoscope according to the third embodiment is a capsule-type device which is completely introduced into the body cavity of a human subject.

That is, an endoscope 40 according to this embodiment is a capsule endoscope, has hemispherical transparent covers 41 fixed at the opposite ends of an organic EL element 10 including an organic light emitting layer 22 as shown in FIG. 8, and is formed into a capsule shape as a whole. The inside of the organic EL element 10 is sealed with the transparent covers 41.

Moreover, an illuminating device 4 according to this embodiment is also provided with a light extracting portion 12 at the base end of the organic EL element 10 including the organic light emitting layer 22, and the light extracting portion 12 extracts a second light L2 proceeding in an in-plane direction from the base end and emits the second light to the rear side. Accordingly, this embodiment can perform a simultaneous illuminating operation of the front and of the side.

In the organic EL element 10, a rear imaging unit 42 is provided for imaging an area which is illuminated at the rear side. The rear imaging unit 42 is composed of an objective lens group 26 and a solid-state imaging element 27 as in an imaging unit 5 and is disposed so as to be opposed to the imaging unit 5 in a back-to-back manner.

Since the endoscope 40 configured as described above has a capsule shape, the endoscope 40 can be introduced into a body cavity while markedly reducing the burden on the human subject.

This embodiment can illuminate both of the front side and the rear side at the same time. In addition, the imaging unit 5 images the brightly illuminated front side and the rear imaging unit 42 images the brightly illuminated rear side. Accordingly, this embodiment can reduce the number of components without the need for a separate arrangement of the front and rear illuminating elements.

Although FIG. 8 shows an example in which the imaging unit 5 and the rear imaging unit 42 protrude outward from the illuminating device 4, the invention is not limited to this. The rear imaging unit 42 is not a necessary configuration and only the imaging unit 5 for imaging the front side may be provided alone. As in the second embodiment, the side light extracting portion 31 may be provided to farther illuminate the side.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

For example, in the above-described embodiments, the medical endoscope imaging the inside of the body cavity of a human subject has been exemplified and described. However, for example, the invention may be applied to an industrial endoscope imaging the inside of a unit, such as an engine, as an object to be observed.

Due to the endoscope according to the invention, a light intensity sufficient to perform imaging can be ensured and thus a subject can be clearly imaged. Moreover, since the diameter of the endoscope according to the invention can be reduced, the burden on the human subject can be reduced.

What is claimed is:

1. An endoscope, at least a part of which is introduced into an object to be observed to image an internal subject, the endoscope comprising:
an illuminating device which illuminates the front side and is disposed so that a normal line is perpendicular to an introduction direction; and
an imaging unit which images the front side and is disposed parallel to the illuminating device,
wherein the illuminating device includes an organic light emitting layer which is interposed between two electrodes, a light shielding portion which blocks first light proceeding in a direction of the normal line among the light generated from the organic light emitting layer, and a light extracting portion which is provided at the tip end of the organic light emitting layer and extracts second light proceeding in an in-plane direction of the organic light emitting layer from the tip end among the generated light to emit the second light to the front side.

2. The endoscope according to claim 1,
wherein the imaging unit is disposed to protrude further forward from the illuminating device than the light extracting portion.

3. The endoscope according to claim 1,
wherein the imaging unit is disposed to retreat further backward from the illuminating device than the light extracting portion.

4. The endoscope according to claim 1,
wherein the illuminating device includes a side light extracting portion for extracting the first light to emit the first light to the side.

5. The endoscope according to claim 4, further comprising;
a side imaging unit for imaging an area illuminated with the first light.

6. The endoscope according to claim 1,
wherein the organic light emitting layer is formed into a cylindrical shape so as to surround the imaging unit.

7. The endoscope according to claim 6,
wherein transparent covers for sealing the inside of the organic light emitting layer are provided at opposite ends of the organic light emitting layer to form the endoscope into a capsule shape as a whole.

8. The endoscope according to claim 7,
wherein the light extracting portion is also provided at the base end of the organic light emitting layer to extract the second light from the base end and thereby emit the second light to the rear side, and
a rear imaging unit for imaging an area illuminated at the rear side is provided in the organic light emitting layer.
